(19) United States
(12) Patent Application Publication
(10) Pub. No.: US 2008/0077177 A1
(43) Pub. Date: Mar. 27, 2008

(54) Hand-assisted laparoscopy forceps

(76) Inventor: Nathan Goldin, Chesapeake, VA (US)

Correspondence Address: BRADLEY D. GOLDIZEN, 505 SOUTH INDEPENDENCE BOULEVARD, SUITE 102, VIRGINIA BEACH, VA, 23452, US

(21) Appl. No.: 11/527,432

(22) Filed: Sep. 26, 2006

(51) Int. Cl. A61B 17/50

(52) U.S. Cl. 606/210

(57) ABSTRACT

A medical instrument for use in surgical procedures such as laparoscopic surgery includes a flexible coupler that secures the instrument to the base of a surgeon's hand or wrist. The flexible coupler assists in securing the instrument within the hand by allowing providing a means of tensioning the device within the hand of the surgeon. In this manner, the medical instrument may be used within the patient. The instrument is small enough to be easily passed through an opening in the abdomen of a patient while being held within or against the palm of the hand. The instrument includes two branches connected together via a base. Each branch includes a jaw, an exterior gripping surface, and a working end arranged opposite the base. Teeth may be provided on an interior surface of each jaw.

There are no related patent applications.

The subject matter of the present invention did not receive federal government research and development funding.

### BACKGROUND OF THE INVENTION

The present invention generally relates to a medical device such as handheld forceps for use in surgical procedures particularly laparoscopic surgeries. More particularly, the invention is directed towards a handheld forceps or clamp sized for use in hand-assisted laparoscopic surgical procedures and includes a flexible attachment means that couples the forceps or clamp to the hand or wrist of the surgeon performing the surgical procedures. The flexible attachment means operates as a retrieval means, should the medical device slip from between the fingers and thumb of the surgeon. Moreover, the present invention is shorter in length than many existing forceps and may advantageously be inserted into a patient's abdomen through a closed or flattened hand.

There are many type of clamps and forceps that are presently used in surgical procedures. Most of these are very large and are designed for use in open environments above the body organ that is the subject of the surgical procedure. The size of traditional forceps and clamps are typically larger than the size of a surgeon's hand. Thus, these forceps or clamps cannot be held fully within the palm of the hand or between the thumb and forefinger when the hand is closed. Moreover, conventional forceps cannot be inserted into a patient's abdomen for use in hand-assisted laparoscopic surgery. The overall shape and length of the present invention allows the forceps to be easily manipulated without inadvertently dropping them into the patient by providing adequate gripping surface along the working jaws of the forceps. Moreover, conventional forceps are not designed to be coupled to the wrist of the surgeon.

It should be recognized that most surgeries are performed with the performing medical personnel wearing rubber gloves. An exterior surface of these gloves may become slick when subjected to body fluids released during surgery. Until now, the overall length of most forceps has been long enough to allow for the adequate gripping of them during surgery.

Conventional surgery performed on the internal organs that are arranged in an abdomen or pelvic region of a patient have required long incisions. These incisions sometimes extend from the pelvic region upwards to the chest cavity and across the entire width of the torso. Large incisions may require the spreading or cutting of a large number of muscle fibers. Thus, conventional surgery is invasive and tends to lead to soreness and extended hospital stays necessary for patient recovery.

In recent years, it has been recognized that most surgeries may be performed in less invasive manners than conventional surgeries. Thus, the idea of laparoscopic surgery, or “keyhole” surgery, has gained much favor in the medical community. In this surgical procedure, ports or sealed openings that extend through the abdominal wall of the patient are provided. A laparoscope and other instruments are inserted through these ports. A pneumoperitoneum is created by the surgical team by insufflating the patient's abdomen with carbon dioxide. The laparoscope is inserted through a small port in a patient's abdomen for viewing the interior of the abdomen; specialized medical instruments are than extended through other small ports to perform a necessary surgery without cutting large openings in the patient. These specialized medical instruments include small working ends that extend through the ports and actuating ends that are arranged on the exterior of the patient. Many surgeons have found it difficult to manipulate the tools thus it is preferable to perform hand-assisted laparoscopic surgery.

In hand-assisted laparoscopic surgery, the surgeon inserts his non-dominate hand into the patient's abdomen to facilitate the dissection. A small opening that is only large enough to accommodate the surgeon's hand is cut into the abdomen. A hand port having a sealed membrane is arranged in the small opening and the surgeon's hand is inserted through this hand port. Often the surgeon will insert his hand in a closed fist style through the hand port. The surgeon then performs the surgery with his hand inserted in the patient. Large conventional forceps and clamps cannot be concealed within the hand of the surgeon as it is slipped through the hand port. Moreover, because of their length, the surgeon risks a chance of puncturing other healthy tissue or organs should traditional forceps be used. The intent of performing laparoscopic surgery is to limit the size of the opening(s) when performing the operation. Thus, ordinary surgical instruments are typically too large to facilitate the operation.

The present applicant is a surgeon who recognizes that there is a heartfelt need for the present device in the medical community. There exists a need for small forceps and/or clamps that can be inserted into a patient's abdomen through a closed or flattened hand. The forceps must be short in length and small in size moreover they must be easily retrieved when the surgeon's hand is withdrawn from the patient. A surgeon must still be able to adequately use the forceps without losing them inside the patient.

The present invention fulfills this heartfelt need in the area of surgery and facilitates the practicing of hand-assisted laparoscopic surgery by providing a device that is specifically designed for performing such surgery.

### SUMMARY OF THE INVENTION

The present invention includes a surgical device that easily fits within the hand of the surgeon and may be secured within the palm of the hand for insertion into a patient. The surgical device includes a forceps device that includes two branches coupled together by a base. Each branch is spring-biased away from one another. A jaw is included on an interior surface of each branch on a working end opposite the base. A grip is provided on an exterior surface of each branch between the working end and base.

An opening is included in the base of the device. A flexible attachment means couples the forceps to the base of the hand or wrist of the surgeon performing the surgical procedures. The flexible attachment means is replaceable and passes through the opening in the base of the device. The flexible attachment means includes an opening that is large enough to accept the hand of the surgeon and may be routed across the surgeon's palm or an exterior surface of the surgeon's hand across the fleshy region between the thumb and forefinger.

The flexible attachment means operates as a retrieval means should the device slip from between the fingers and thumb of the surgeon. Moreover, the present invention is shorter in length than existing forceps and may advantageously be inserted into a patient's abdomen through a closed or flattened hand.

It is an object of the invention to provide a medical device having an opening through which a flexible attachment means extends to couple the medical device to the wrist of a surgeon.

It is another object of the invention to provide a flexible attachment means that is quickly removed from the wrist of the surgeon and replaced during surgery.

It is a further object of the invention to provide a flexible attachment means that may be routed across the palm of the surgeon to be coupled to the wrist. Alternatively, the flexible attachment means may be routed across an exterior of the hand across the fleshy part that exists between the thumb and forefinger.

It is an addition object of the invention to provide a flexible attachment means that includes an opening through which the surgeon's hand passes such that the forceps are coupled to the wrist of a surgeon. As the surgeon performs parts of the dissection not requiring forceps, the instrument may be released into the abdominal cavity of the patient while remaining attached to the surgeon's wrist via the flexible attachment means or lanyard. The forceps are easily retrieved when needed and will not be inadvertently left in the patient.

It is a further object of the invention to teach a medical device that is a forceps or clamps for use in hand-assisted laparoscopic surgery.

It is an object of the invention to provide a laparoscopic forceps that comprises an opening through which a flexible attachment means extends to couple the laparoscopic forceps to the wrist of a surgeon.

Additional objects and advantages of the invention will be set forth in part in the description which follows, and in part will be obvious from the description, or may be learned from practicing the invention. The objects and advantages of the invention will be obtained by means of instrumentalities in combinations particularly pointed out in the appended claims.

### BRIEF DESCRIPTION OF THE DRAWINGS

FIG. 1 is a perspective view of a first embodiment of the medical device shown without the flexible attachment lanyard.

FIG. 2 is a perspective view of a second embodiment of the medical device shown without the flexible attachment lanyard.

FIG. 3A is a perspective view of the second embodiment of the invention comprising a flexible attachment including an opening through which a surgeon passes his hand to couple the medical device to the surgeon's wrist. FIG. 3B is a cross section view of FIG. 3A.

FIG. 4 is a perspective view of the invention and showing with the flexible attachment securing the medical device to a hand of a surgeon in which the device is being held.

FIG. 5 is an alternative view showing the surgeon inserting the medical device into the abdominal region of the patient via a closed hand that forms a fist.

FIG. 6 depicts a medical device that has been retrieved from a patient after slipping from the surgeon's fingers.

FIG. 7 is a first view of the medical device being tensioned with the ring finger and the small finger to resist slippage while performing surgery.

FIG. 8 is another view showing the medical instrument being held across the palm of the surgeon's hand.

FIG. 9 is an additional view showing the medical instrument being held in a closed hand. This depicts a method for introducing the medical instrument into the patient's abdomen.

FIG. 10 is an alternative view showing the flexible attachment being routed across the fleshy region between the thumb and forefinger.

### DETAILED DESCRIPTION OF THE INVENTION

The embodiments of the invention and the various features and advantageous details thereof are more fully explained with reference to the non-limiting embodiments and examples that are described and/or illustrated in the accompanying drawings and set forth in the following description. It should be noted that the features illustrated in the drawings are not necessarily drawn to scale, and the features of one embodiment may be employed with the other embodiments as the skilled artisan recognizes, even if not explicitly stated herein. Descriptions of well-known components and techniques may be omitted to avoid obscuring the invention. The examples used herein are intended merely to facilitate an understanding of ways in which the invention may be practiced and to further enable those skilled in the art to practice the invention. Accordingly, the examples and embodiments set forth herein should not be construed as limiting the scope of the invention, which is defined by the appended claims. Moreover, it is noted that like reference numerals represent similar parts throughout the several views of the drawings.

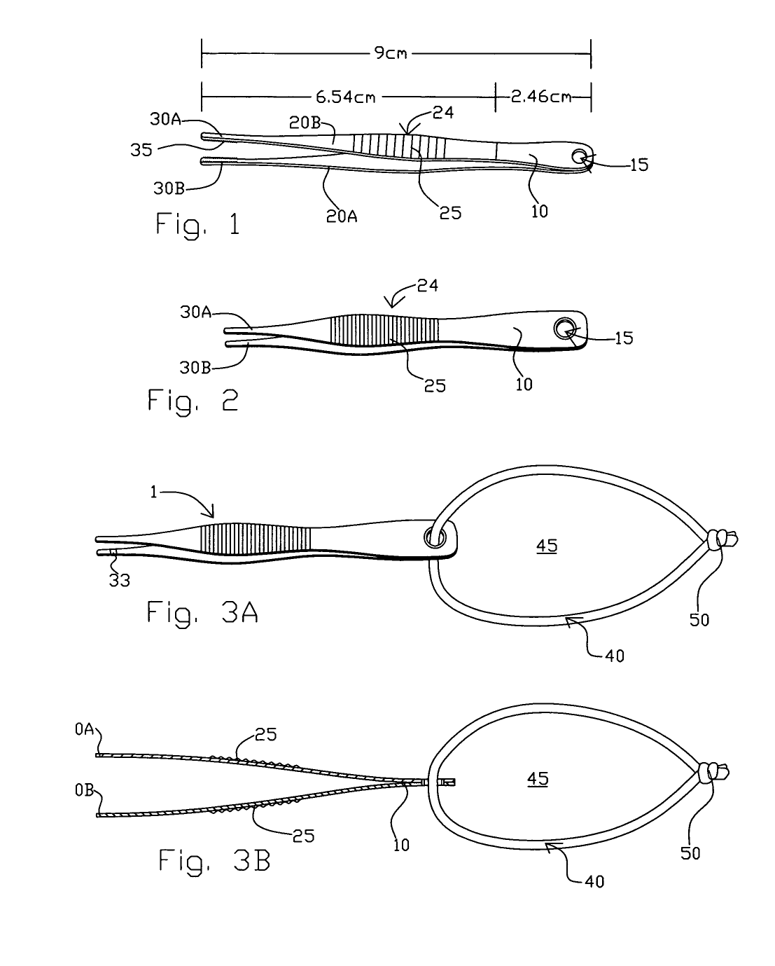

A pair of forceps are a hand-held instrument that is used for grasping items. In surgical procedures, forceps are used to handle tissues, needles and dressings. FIG. 1 and 2 show respective embodiments of the laparoscopic forceps 1, without a flexible attachment means 40. The overall length of the forceps is substantially 9 cm as shown in FIG. 1. The forceps 1 may be formed from stainless steel, medical grade metal, composite material, plastic or the like.

The pair of forceps 1 includes a base 10 having an opening 15 for accepting a flexible attachment means 40, as shown in FIGS. 3A through 10. The base 10 is substantially 2.46 cm in length. Two branches 20A and 20B, collectively referred to as 20 herein, extend from the base 10 which couples them together. The branches 20 are biased away from one another and formed as shown in FIG. 3B. Each branch 20 includes an enlarged region 24 that comprises an area having a larger width that adjoining regions of the branch. This is the grip where the surgeon applies pressure to close the branches that form the working jaws. Undulations 25 are provided on the exterior surface of each branch and along this enlarged region 24 to provide an area where the surgeon directs pressure to operate the forceps 1 to grip tissue and the like. The undulations 25 create a roughened area that prevents the surgeon's gloves from inadvertently slipping from the forceps during use. It is to be understood by the skilled artisan that while the figures only depict undulations, the exterior surface of each branch may be provided with scoring or the like to roughen it. The length of each branch is substantially 6.54 cm.

Working ends 30A and 30B, collectively referred to as 30 hereinafter, are arranged at ends of the branches 20 opposite the base 10. In the first embodiment shown in FIG. 1, interior surfaces of the working ends 30 include teeth 35 arranged there along. It is notable that the working ends 30 have a smaller width than any other region of the branches. The teeth are not present in the second embodiment shown in FIG. 2. The first embodiment is useful for grasping tissue. The second embodiment may be used for gripping needles and may include a needle groove 33 for accommodating needles during suturing processes.

A flexible attachment means 40 passes through opening 15 as shown in FIG. 3. The flexible attachment means 40 includes a hand opening 45 through which the surgeon passes his hand to couple the forceps 1 to his wrist. The flexible attachment means may be severed and replaced as necessary. Moreover, the flexible attachment: may comprise one or more selected from a group consisting of surgical thread, rubber, or other inert material.

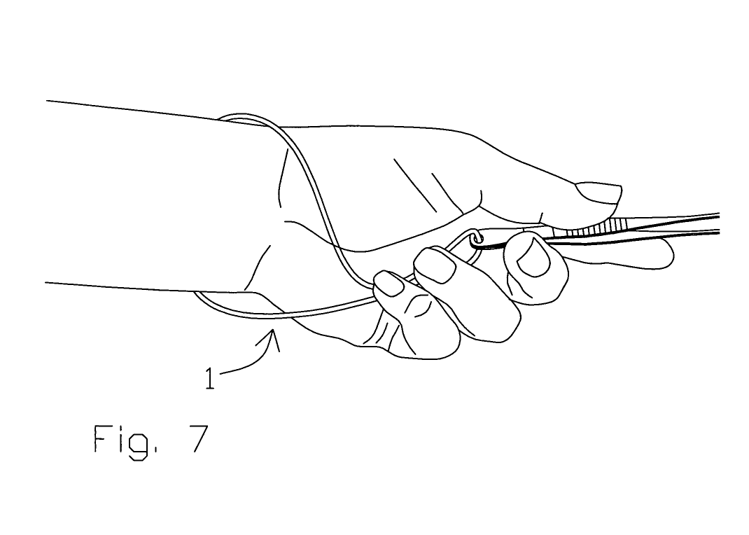

The forceps include a short length that may be inadequate for properly holding the instrument without the coupler attached. However, the flexible attachment means extends the gripping region of the device such that the third through fifth fingers of the surgeon's hand may be wrapped around the flexible attachment as shown in FIG. 7. If the external diameter of the flexible attachment means small, then tension may be applied to the base of the instrument to securely seat the instrument between the thumb and forefinger. Moreover, the overall length of the coupler may be varied to allow for more or less tensioning of the base within the hand. For example, the length of the flexible attachment means between the wrist of the surgeon and the base of the instrument may be short such that only the third finger of the surgeon's hand exerts pressure against the flexible attachment means. Alternatively the length of a new flexible attachment means may be increased to accommodate the fourth and fifth fingers to increase tension on the flexible attachment means. Since suturing materials may be utilized as the flexible attachment means, its length may be easily varied according to a surgeon's preference.

In addition to the flexible attachment including a fastening means necessary for helping to hold the instrument in place when it is used, The flexible attachment also provides an easy retrieval device and method should the device slip from the surgeons hand during surgery. The surgeon withdraws his hand from within the patient and the instrument is retrieved. Moreover, the surgeon may intentionally allow the device to slip from his fingers without losing the device in the patient's abdomen.

The flexible attachment means may be ran across the palm of the hand and grasped by the middle, ring and/or small finger of the surgeon to aid in controlling or manipulating the forceps. Alternatively, the forceps may be coupled to the wrist by running the flexible attachment means across the exterior of the hand between the surgeon's thumb and forefinger.

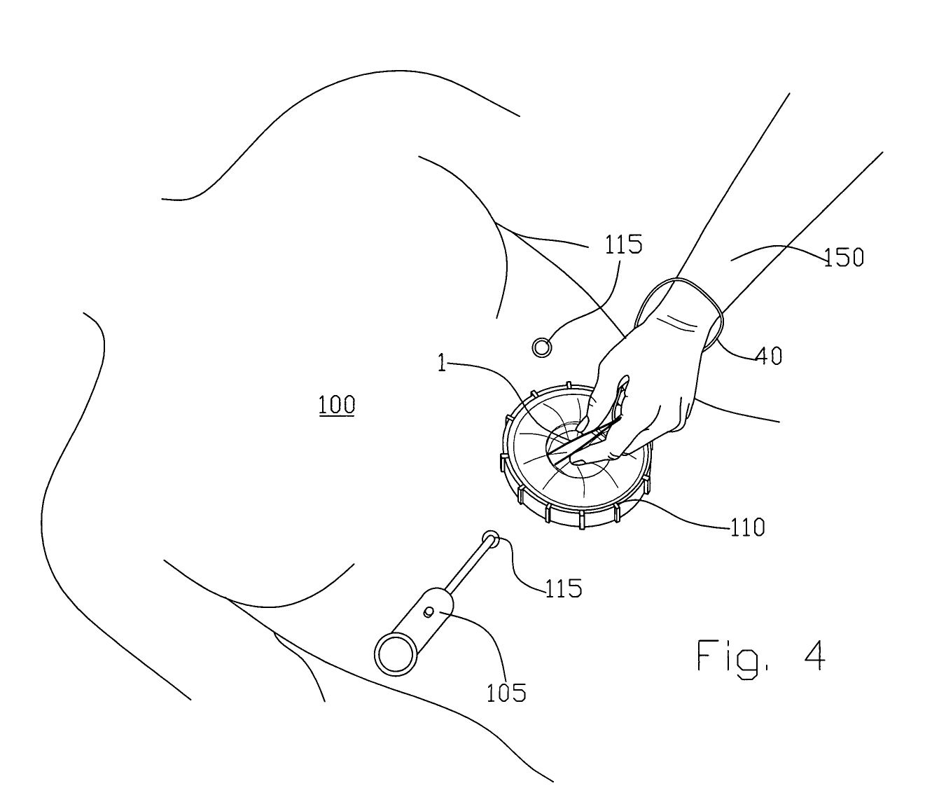

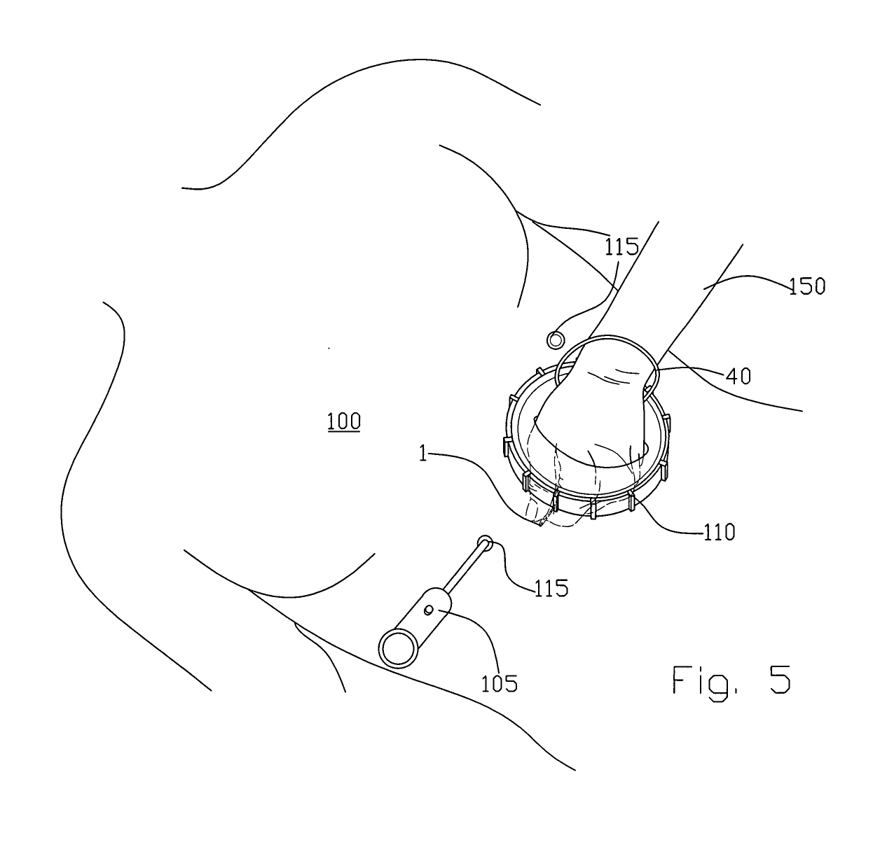

FIG. 4 depicts a patient 100 upon whom hand-assisted laparoscopic surgery is being performed. For ease in understanding the invention, the figures depict an ungloved surgeon's hand and some of the necessary elements for performing laparoscopy are not shown. For example, it is to be understood that surgical gloves are worn during the surgery. More importantly, a seal must be maintained in the patient's abdomen to create pneumoperitoneum. Thus, it is necessary for an acceptable hand port 110 to be provided. A scope 105 is inserted through a port 115. The forceps 1 are then secured around the wrist of the surgeon 150. Other instruments may be inserted through the other shown port 115.

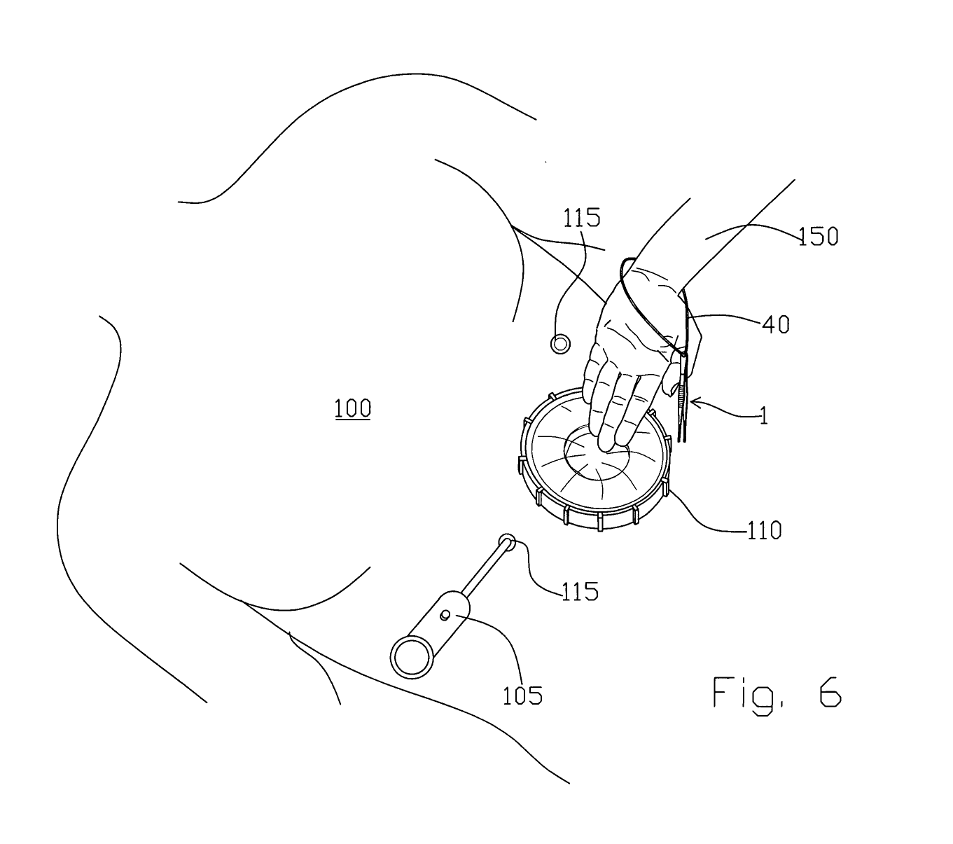

The forceps 1 may be introduced into the patient's abdomen through a closed hand as shown in FIG. 5. As can be recognized, the surgeon may allow the forceps 1 to drop from his hand as necessary during surgery. As shown in FIG. 6, the forceps 1 may be easily retrieved by withdrawing the hand through the hand port 110.

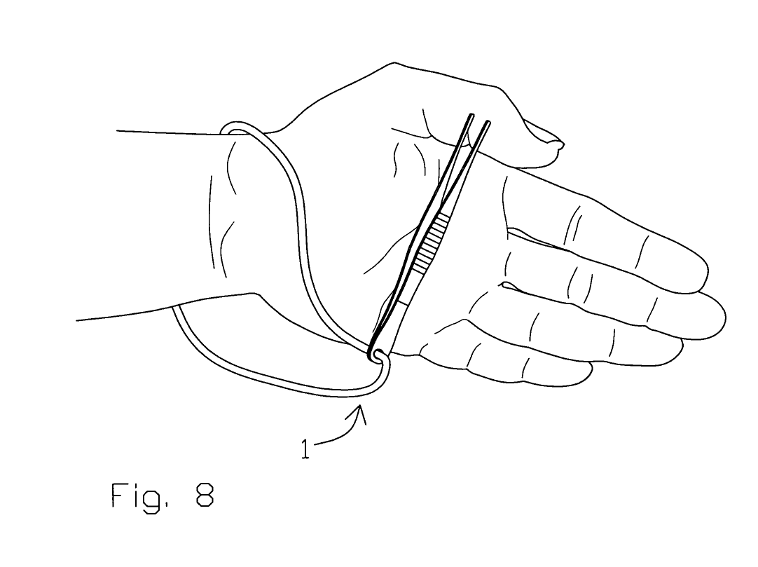

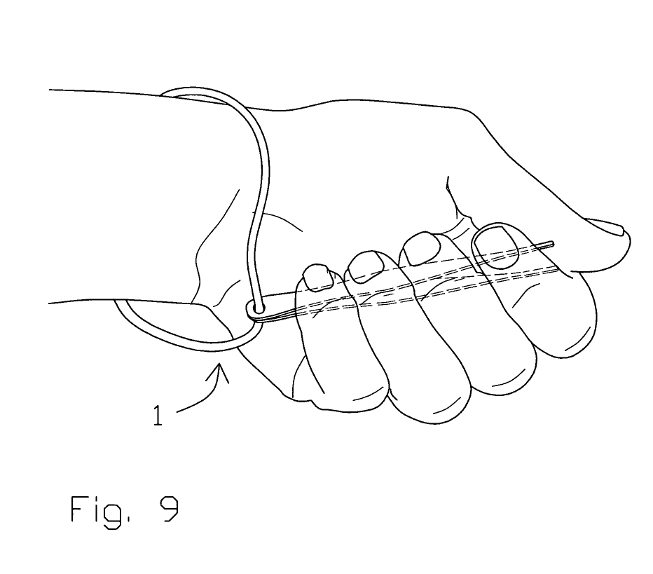

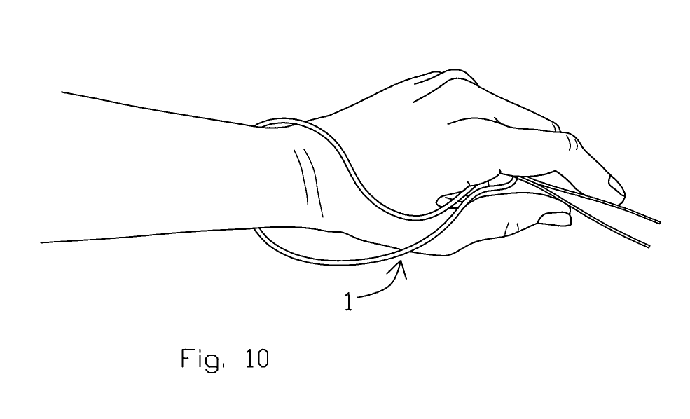

FIG. 8 shows the forceps in the palm of the surgeons hand along with the flexible attachment means. As clearly depicted in FIG. 9, the forceps may be substantially retained within the closed fist of the surgeon. FIG. 10 depicts an alternative way of routing the flexible attachment means across the fleshy part of the surgeon's hand.

While the invention has been described with respect to preferred embodiments, it is intended that all matter contained in the above description or shown in the accompanying drawings shall be interpreted as illustrative and not in limiting sense. From the above disclosure of the general principles of the present invention and the preceding detailed description, those skilled in the art will readily comprehend the various modifications to which the present invention is susceptible. Therefore, the scope of the invention should be limited only by the following claims and equivalents thereof.

I claim:

1. A pair of laparoscopic forceps comprising:
a base having a hole that extends through the base;
a first branch extending from said base and having an inner and an outer surface area, said first branch having a working end arranged opposite the base;
a second branch extending from said base and being parallel to said first branch, said second branch having an inner and an outer surface area, said second branch having a working end arranged opposite the base; and
a flexible attachment means that passes through said base and includes an opening through which a surgeon's hand passes.

2. The pair of laparoscopic forceps of claim 1 wherein said first and second branches include teeth arranged on an inner surface of each branch at the working end.

3. The pair of laparoscopic forceps of claim 1 wherein an overall length of the forceps is substantially 9 cm.

4. The pair of laparoscopic forceps of claim 3 wherein a length of the base is 2.46 cm and a length of each branch is 6.54 cm.

5. The pair of laparoscopic forceps of claim 1 wherein said flexible attachment means comprises one or more selected from a group consisting of a suture of medical thread, a rubber band, and a lanyard.

6. The forceps of claim 1 formed from one or more selected from a group consisting of stainless steel, medical grade metal, composite material, and plastic.

7. The forceps of claim 1 wherein each branch includes an enlarged region that comprises an area having a larger width than adjoining regions of the branch.

8. The forceps of claim 1 wherein the exterior surfaces of the branches comprise undulations that provide an area where the surgeon directs pressure to operate the forceps to grip tissue.

9. A pair of laparoscopic forceps comprising:
a base having an opening that extends through the base;
a pair of parallel branches extending from said base, each parallel branch having an inner surface, an outer surface area and a working end arranged opposite the base;
a flexible attachment means that passes through opening that extends through said base, said flexible attachment means including an opening through which a surgeon's hand passes.

10. The pair of laparoscopic forceps of claim 1 wherein the pair of parallel branches include teeth arranged on an inner surface of each branch at the working end.

11. The pair of laparoscopic forceps of claim 9 wherein an overall length of the forceps is substantially 9 cm.

12. The pair of laparoscopic forceps of claim 11 wherein a length of the base is 2.46 cm and a length of each branch is 6.54 cm.

13. The pair of laparoscopic forceps of claim 1 wherein said flexible attachment means comprises one or more selected from a group consisting of a suture of medical thread, a rubber band, and a lanyard.

14. The forceps of claim 9 formed from one or more selected from a group consisting of stainless steel, medical grade metal, composite material, and plastic.

15. The forceps of claim 9 wherein each branch includes an enlarged region that comprises an area having a larger width than adjoining regions of the branch.

16. The forceps of claim 9 wherein the exterior surfaces of the branches comprise undulations that provide an area where the surgeon directs pressure to operate the forceps to grip tissue.

17. A pair of laparoscopic forceps comprising:
a base having a hole that extends through the base;
a first branch extending from said base and having an inner and an outer surface area, said first branch having a working end arranged opposite the base;
a second branch extending from said base and being parallel to said first branch, said second branch having an inner and an outer surface area, said second branch having a working end arranged opposite the base; and
a flexible attachment means that passes through said base and includes an opening through which a surgeon's hand passes;
wherein the exterior surfaces of the branches comprise undulations that provide an area where the surgeon directs pressure to operate the forceps to grip tissue.

18. The pair of laparoscopic forceps of claim 17 wherein an overall length of the forceps is substantially 9 cm.

19. The pair of laparoscopic forceps of claim 18 wherein a length of the base is 2.46 cm and a length of each branch is 6.54 cm.

20. The pair of laparoscopic forceps of claim 17 wherein said flexible attachment means comprises one or more selected from a group consisting of a suture of medical thread, a rubber band, and a lanyard.
